(19) United States
(12) Patent Application Publication
(10) Pub. No.: US 2019/0105493 A1
(43) Pub. Date: Apr. 11, 2019

(54) REMOTE HEALTH CARE SYSTEM

(71) Applicant: Y-BRAIN INC., Daejeon (KR)

(72) Inventor: Kiwon LEE, Gyeonggi-do (KR)

(21) Appl. No.: 15/833,043

(22) Filed: Dec. 6, 2017

(30) Foreign Application Priority Data
Oct. 10, 2017 (KR) 10-2017-0128930

(51) Int. Cl. A61N 1/20; A61N 1/02

(52) U.S. Cl. CPC A61N 1/20; A61N 1/025

(57) ABSTRACT

A remote health care system comprising: a stimulation module including: a control unit for storing inputted stimulation strength, stimulation duration and stimulation frequency in internal memory, and a current providing unit for providing the control unit with current corresponding to the stimulation strength, the stimulation duration and the stimulation frequency, in which the control unit executes stimulation corresponding to the stimulation strength, the stimulation duration and the stimulation frequency using the current provided by the current providing unit; and a management station for inputting the stimulation strength, the stimulation duration and the stimulation frequency into the stimulation module.

### CROSS REFERENCE

This application claims foreign priority under Paris Convention to the Korean Patent Application No. 10-2017-0128930 filed Oct. 10,2017 with the Korean Intellectual Property Office, the entire contents of which are incorporated herein by reference.

### BACKGROUND

The present invention relates to a remote health care system, and more specifically, to a remote health care system, which can store stimulation strength, stimulation duration and stimulation frequency inputted through a management station and easily execute stimulation at a stimulation location (e.g., home of a patient) convenient to execute stimulation corresponding to the stimulation strength, the stimulation duration and the stimulation frequency.

Presently, brains are stimulated in a variety of methods including deep electrical stimulation, Transcranial Magnetic Stimulation (TMS), Transcranial Electrical Stimulation (TES), Transcranial Direct Current Stimulation (tDCS) and Transcranial Random Noise Stimulation (tRNS).

Particularly, the Transcranial Direct Current Stimulation is utilized as a method of stimulating a brain in a rehabilitation or neuropsychiatry clinic, and it is reported to be effective for improving cognitive ability and treating mental disorders such as depression, Attention Deficit Hyperactivity Disorder (ADHD) and the like.

A background technique of the present invention is disclosed in US Laid-opened Patent No. US 2009/0287108.

### SUMMARY OF THE INVENTION

Therefore, the present invention has been made in view of the above problems, and it is an object of the present invention to provide a remote health care system, which can store stimulation strength, stimulation duration and stimulation frequency inputted through a management station and easily execute stimulation at a remote location (e.g., home of a patient) convenient to execute stimulation corresponding to the stimulation strength, the stimulation duration and the stimulation frequency.

To accomplish the above object, according to one aspect of the present invention, there is provided a remote health care system comprising: a stimulation module including: a control unit for storing inputted stimulation strength, stimulation duration and stimulation frequency in internal memory, and a current providing unit for providing the control unit with current corresponding to the stimulation strength, the stimulation duration and the stimulation frequency, in which the control unit executes stimulation corresponding to the stimulation strength, the stimulation duration and the stimulation frequency using the current provided by the current providing unit; and a management station for inputting the stimulation strength, the stimulation duration and the stimulation frequency into the stimulation module.

In the remote health care system according to an embodiment of the present invention, the stimulation strength may be strength of DC current, the stimulation duration may be a time period of sustaining the strength of DC current, and the stimulation frequency may be the number of times of repeating the time period of sustaining the strength of DC current.

The stimulation module of the remote health care system according to an embodiment of the present invention further may include a real-time clock for transferring real-time to the control unit, and if the number of times of stimulation is two or more, the control unit may control to execute second and subsequent stimulations after a minimum time period is elapsed using the real-time.

The stimulation module of the remote health care system according to an embodiment of the present invention may further include an alarm unit controlled by the control unit to inform a recommended time desirable to execute the stimulation using the real-time.

In the remote health care system according to an embodiment of the present invention, if the stimulation module is linked to a mobile application, the stimulation module may control the alarm unit to inform a stimulation execution schedule time set through the mobile application.

The stimulation module of the remote health care system according to an embodiment of the present invention may further include a memory unit controlled by the control unit to store a time when the stimulation has been executed using the real-time.

The stimulation module of the remote health care system according to an embodiment of the present invention may be controlled by the control unit to store stimulation execution history configured of the stimulation strength, the stimulation duration, the stimulation frequency and the stimulation execution time in the memory unit.

The stimulation module of the remote health care system according to an embodiment of the present invention may further include a display unit controlled by the control unit to separately display the number of times of stimulation that has been executed already and the number of times of stimulation that has not been executed yet or to display remaining stimulation duration.

The management station of the remote health care system according to an embodiment of the present invention may search for stimulation execution history from the stimulation module.

When the stimulation module of the remote health care system according to an embodiment of the present invention approaches the management station within a Bluetooth communication distance, the stimulation execution history stored in the memory unit of the stimulation module may be transferred to and stored in the management station in a Bluetooth Low Energy (BLE) method.

The remote health care system according to an embodiment of the present invention may further comprise a charging unit for charging the stimulation module.

### BRIEF DESCRIPTION OF THE DRAWINGS

FIG. 1 is a view showing the configuration of a remote health care system according to an embodiment of the present invention.

FIG. 2 is a block diagram showing a stimulation module of a remote health care system according to an embodiment of the present invention.

FIG. 3 is a view showing the display unit of the stimulation module of FIG. 1.

### DETAILED DESCRIPTION OF THE PREFERRED EMBODIMENT

Specific matters of other embodiments are included in the detailed description and the drawings.

Advantages and features of the present invention and a method of achieving the same will be more clearly understood from the embodiments described below with reference to the accompanying drawings. However, the present invention is not limited to the following embodiments but may be implemented in various different forms. The embodiments are provided merely to complete disclosure of the present invention and to fully provide a person having ordinary skill in the art to which the present invention pertains with the category of the invention. The invention is defined only by the category of the claims. Wherever possible, the same reference numbers will be used throughout the specification to refer to the same or like parts.

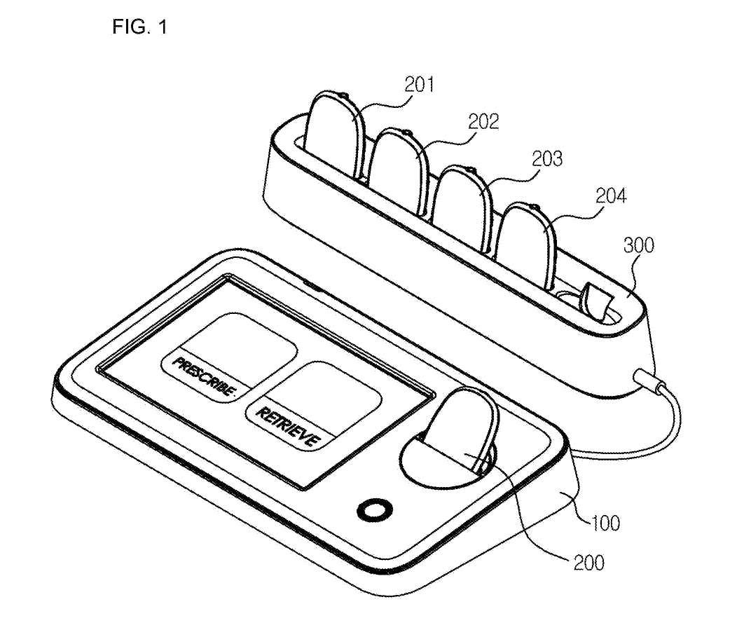

In a remote health care system according to an embodiment of the present invention, as shown in FIG. 1, while a stimulation module 200 is loaded on a management station 100, a doctor in charge selects the PRESCRIBE mode and inputs stimulation strength, stimulation duration and stimulation frequency into the loaded stimulation module 200.

In addition, the remote health care system according to an embodiment of the present invention may further include a charging unit 300 capable of simultaneously charging a plurality of stimulation modules 201, 202, 203 and 204 as shown in FIG. 1.

Here, it is natural that load of the stimulation module 200 on the management station 100 or connection between the stimulation modules 201, 202, 203 and 204 and the charging unit 300 is not limited to the direct contact method as shown in the figure and may be accomplished through direct or indirect connection in a general wired or wireless communication method.

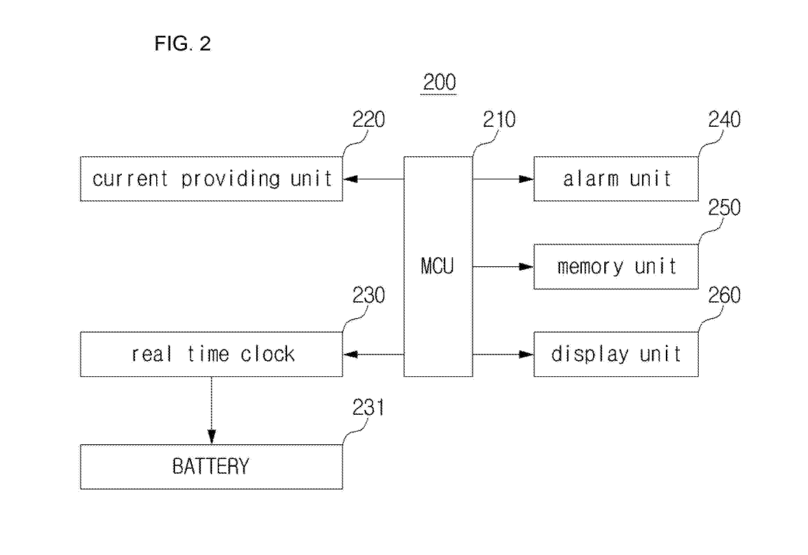

A control unit 210 of the stimulation module 200 is configured of a Micro Controller Unit (MCU) as shown in FIG. 2 and stores the inputted stimulation strength, stimulation duration and stimulation frequency in internal memory.

A current providing unit 220 includes a current sensor and an operational amplifier and provides the control unit 210 with current corresponding to the stimulation strength, the stimulation duration and the stimulation frequency, and the control unit 210 executes stimulation corresponding to the stimulation strength, the stimulation duration and the stimulation frequency using the current provided by the current providing unit 220.

Here, the stimulation strength is strength of DC current, for example, DC current of about 0 to 2 mA, the stimulation duration is a time period of sustaining the strength of DC current, for example, a time period of about ten to thirty minutes, and the stimulation frequency is the number of times of repeating the time period of sustaining the strength of DC current, for example, once a day for thirty days.

For example, if the doctor in charge inputs a prescription such as 2 mA, thirty minutes, once a day and fifteen days into the stimulation module 200 through the management station 100, information like this is stored in the internal memory of a corresponding stimulation module. Accordingly, if a patient is prescribed the corresponding stimulation module 200 and takes it home, home treatment may be performed without separately visiting the clinic for fifteen days of the prescription period.

In addition, since the treatment process performed by the patient himself or herself during the home treatment period like this (e.g., fifteen days) is recorded in the stimulation module 200 again, if the patient returns the stimulation module 200 to the clinic after the treatment period is over, the clinic side may confirm detailed treatment records of the patient through the management station 100.

Describing in detail, if the patient completes the treatment exactly for thirty minutes only for ten days during fifteen days of the home treatment period and stops the treatment in the middle for two days, the management station 100 grasps through the corresponding stimulation module 200 that the patient did not execute the treatment for three days and stopped the treatment in the middle for two days. It is natural to grasp even the detailed information on in which day the treatment has been omitted in this process and, in the case of the stopped treatment, at which minute the treatment has been stopped in the treatment process of total thirty minutes.

Meanwhile, the stimulation module further includes a real-time clock 230 for transferring real-time to the control unit 210, and if the number of times of stimulation is two or more, the control unit 210 may control to execute second and subsequent stimulations after a minimum time period is elapsed using the real-time. Here, the minimum time period may be set to be, for example, four hours or more. For example, if the stimulation strength is DC current of 1 mA, the stimulation duration is twenty minutes, the stimulation frequency is once a day for thirty days, the minimum time period is four hours, and the first stimulation is executed at 23:00 on Sep. 1^{st}, 2016, the second stimulation can be executed only after 03:00 on Sep. 2^{nd}, 2016.

In addition, as described above, the stimulation module 200 may be configured to record and manage corresponding information if the stimulation is not executed during certain one day. In other words, if the stimulation is not executed between 00:00 and 24:00 on September 6^{th} although it is set to execute stimulation thirty times for thirty days between September 1^{st} and 30^{th}, the control unit 210 may record and manage corresponding information. In this case, although the overall stimulation schedule is preferably set to finish on September 30^{th} regardless of the number of times of stimulation that has not been executed, the present invention is not necessarily limited thereto.

Meanwhile, the stimulation module 200 may be provided with a separate battery 231 and supply power to the real-time clock 230.

In addition, the stimulation module further includes an alarm unit 240 controlled by the control unit 210 to inform a recommended time desirable to execute the stimulation using the real-time.

For example, when the stimulation strength is DC current of 1 mA, the stimulation duration is twenty minutes, and the stimulation frequency is once a day for thirty days, a recommended time is informed at 21:00 every day through the alarm unit 240 so that stimulation may be executed at the same time for thirty days.

In addition, the stimulation module further includes a memory unit 250 controlled by the control unit 210 to store a time when the stimulation has been executed using the real-time. Specifically, the control unit 210 controls to store stimulation execution history configured of the stimulation strength, the stimulation duration, the stimulation frequency and the stimulation execution time in the memory unit 250.

Meanwhile, remaining stimulation duration is stored in the memory unit 250 when the stimulation is stopped due to unexpected reasons, and if the stimulation is executed at a later time, the control unit 210 executes remaining stimulation using the remaining stimulation duration stored in the memory unit 250.

For example, when the stimulation strength is DC current of 1 mA, the stimulation duration is twenty minutes, and the stimulation frequency is once a day for thirty days, a time when the stimulation has been executed k stored in the memory unit 250 using the real-time.

Although it is not described in detail in the specification, the stimulation module 200 according to an embodiment of the present invention may have a configuration of directly contacting with the body of the patient using a separate device (e.g., a body attach-type patch, a hairband or the like including electrodes). In relation to the specific configuration of the separate device like this, contents of other applied patents of the inventor may be referenced.

Here, in the remote health care system according to an embodiment of the present invention, as shown in FIG. 1, while the stimulation module 200 is loaded on the management station 100, a doctor in charge may select the RETRIEVE mode and search for stimulation execution history from the memory unit 250 of the loaded stimulation module 200 and stores the stimulation execution history stored in the memory unit 250 of the loaded stimulation module 200 into the management station 100. Although the stimulation execution history may be transferred and stored while the stimulation module 200 is loaded on the management station 100, when the stimulation module 200 approaches the management station 100 within a Bluetooth communication distance, the stimulation execution history stored in the memory unit 250 of the stimulation module 200 may be transferred to and stored in the management station 100 in a Bluetooth Low Energy (BLE) method.

That is, if the stimulation execution history is transferred while the stimulation module 200 is inserted in the management station as shown in the figure, the work may be delayed due to the time or the like consumed therefor, and therefore, it may be configured to automatically perform, when the stimulation module 200 enters a predetermined space in a circle, the process like this through a predetermined confirmation procedure using a wireless communication method based on a general beacon method or the like.

Meanwhile, a display unit 260 controlled by the control unit 210 to separately display the number of times of stimulation that has been executed already and the number of times of stimulation that has not been executed yet or to display remaining stimulation duration may be included in the stimulation module 200.

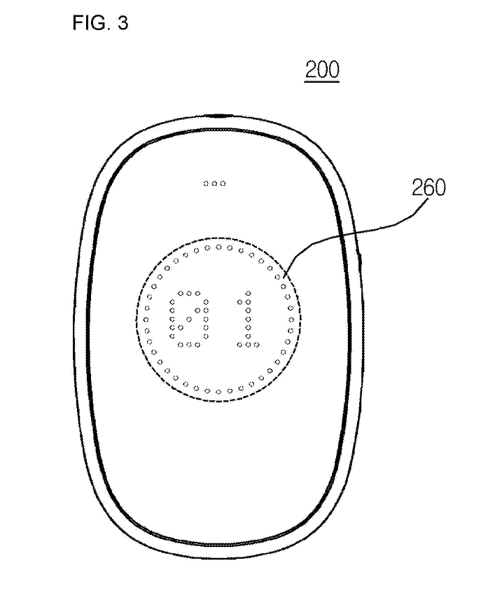

Referring to FIG. 3, for example, if the stimulation strength is DC current of 1 mA, the stimulation duration is twenty minutes, the stimulation frequency is once a day for thirty days, and the first stimulation is executed on Sep. 1^{st}, 2016, ‘1’, which is the number of times of stimulation that has already been executed, is displayed by turning on one blue Light Emitting Diode (LED), and ‘29’, which is the number of times of stimulation that has not been executed yet, is displayed on the display unit 260 by turning on twenty-nine red LEDs, or if stimulation is executed while ‘20’ is displayed on the display unit 260 as the remaining stimulation duration, it is displayed as 19, 18, 17, . . . , 2, 1, 0.

Alternatively, it may be configured such that although information on the remaining number of times of stimulation is displayed in normal times using numeric information of the display unit 260, information on the remaining stimulation duration may be displayed only when the stimulation is executed.

For reference, when the stimulation module 200 of the remote health care system according to an embodiment of the present invention is used for treatment purpose in a clinic, not for home treatment, display of the information on the remaining number of times of stimulation may not be necessary, and accordingly, in this case, it may be configured not to display any information or to display unmeaningful information such as 0 or the like in normal times.

Meanwhile, in the remote health care system according to an embodiment of the present invention, when a stimulation execution schedule time is set through a mobile application by linking the stimulation module 200 to the mobile application, the alarm unit 240 is controlled to inform the stimulation execution schedule time.

In other words, if an application dedicated to the remote health care system is mounted on a mobile terminal such as a smart phone of a patient, the application may perform various functions such as an alarm for executing stimulation, an alarm for taking medicine, notification of information for visiting a clinic and the like, and furthermore, it may provide active information corresponding in real-time to information on the present treatment state through linking or the like to the stimulation module 200. For example, the active information like this may include a message of appreciation and praise provided when scheduled treatment is executed well, an advanced clinic visit schedule automatically notified when the scheduled stimulation treatment is not executed more than a predetermined number of times, or the like.

The remote health care system according to an embodiment of the present invention can store stimulation strength, stimulation duration and stimulation frequency inputted through a management station and easily execute stimulation at a remote location (e.g., home of a patient) convenient to execute stimulation corresponding to the stimulation strength, the stimulation duration and the stimulation frequency.

A person having ordinary skill in the art to which the present invention pertains may understand that the present invention may be embodied in other specific forms without changing its technical spirit or essential features of the present invention.

Therefore, the embodiments described above are to be considered in all respects as illustrative and not restrictive.

The scope of the present invention is represented by the claims rather than the detailed description described above, and it is to be interpreted that the meaning and scope of the claims and all the changes or modified forms derived from the equivalents thereof come within the scope of the present invention.

What is claimed is:

1. A remote health care system comprising:
a stimulation module including: a control unit for storing inputted stimulation strength, stimulation duration and stimulation frequency in internal memory, and a current providing unit for providing the control unit with current corresponding to the stimulation strength, the stimulation duration and the stimulation frequency, wherein the control unit executes stimulation corresponding to the stimulation strength, the stimulation duration and the stimulation frequency using the current provided by the current providing unit; and
a management station for inputting the stimulation strength, the stimulation duration and the stimulation frequency into the stimulation module.

2. The system according to claim 1, wherein the stimulation strength is strength of DC current, the stimulation duration is a time period of sustaining the strength of DC current, and the stimulation frequency is the number of times of repeating the time period of sustaining the strength of DC current.

3. The system according to claim 2, wherein the stimulation module further includes a real-time clock for transferring real-time to the control unit, wherein if the number of times of stimulation is two or more, the control unit controls to execute second and subsequent stimulations after a minimum time period is elapsed using the real-time.

4. The system according to claim 3, wherein the stimulation module further includes an alarm unit controlled by the control unit to inform a recommended time desirable to execute the stimulation using the real-time.

5. The system according to claim 4, wherein if the stimulation module is linked to a mobile application, the stimulation module controls the alarm unit to inform a stimulation execution schedule time set through the mobile application.

6. The system according to claim 3, wherein the stimulation module further includes a memory unit controlled by the control unit to store a time when the stimulation has been executed using the real-time.

7. The system according to claim 6, wherein the stimulation module is controlled by the control unit to store stimulation execution history configured of the stimulation strength, the stimulation duration, the stimulation frequency and the stimulation execution time in the memory unit.

8. The system according to claim 3, wherein the stimulation module further includes a display unit controlled by the control unit to separately display the number of times of stimulation that has been executed already and the number of times of stimulation that has not been executed yet or to display remaining stimulation duration.

9. The system according to claim 7, wherein the management station searches for stimulation execution history from the stimulation module.

10. The system according to claim 7, wherein when the stimulation module approaches the management station within a Bluetooth communication distance, the stimulation execution history stored in the memory unit of the stimulation module is transferred to and stored in the management station in a Bluetooth Low Energy (BLE) method.

11. The system according to claim 2, further comprising a charging unit for charging the stimulation module.
